(19) United States
(12) Patent Application Publication
(10) Pub. No.: US 2026/0022297 A1
(43) Pub. Date: Jan. 22, 2026

(54) Phage and Its Use in Compensating Soil Nitrogen Fixation

(71) Applicants: NANJING AGRICULTURAL UNIVERSITY, Jiangsu (CN); Institute of Soil Science, Chinese Academy of Sciences, Jiangsu (CN)

(72) Inventors: Mingming SUN, Jiangsu (CN); Wen ZHANG, Jiangsu (CN); Shuyue LIU, Jiangsu (CN); Mao YE, Jiangsu (CN); Yunling WU, Jiangsu (CN); Shimao WU, Jiangsu (CN); Yaru WU, Jiangsu (CN); Tianjing YU, Jiangsu (CN); Feng HU, Jiangsu (CN)

(21) Appl. No.: 19/070,325

(22) Filed: Mar. 4, 2025

(30) Foreign Application Priority Data
Jul. 21, 2024 (CN) 202410978417.9

(51) Int. Cl. C09K 17/14; C09K 101/00; C12N 15/74

(52) U.S. Cl. CPC C09K 17/14; C12N 15/74; C09K 2101/00; C12N 2795/00043

(57) ABSTRACT

A phage and its use in compensating soil nitrogen fixation: Klebsiella pneumoniae phage φYSZKA, wherein the method has solved some problems of conventional azotobacters, such as high cost, interspecific competition and limited range of application. Through phage transplantation, the efficiency of microbial nitrogen fixation in soil can be improved, and thus, more nitrogen sources may be provided for the nitrogen cycle and plant growth in ecosystems. Also, a new solution is provided for the nitrogen sequestration process of soil in saline-alkali lands, tea plantations and pesticide-polluted sites in China.

### BACKGROUND OF THE INVENTION

The present invention belongs to the technical field of phages, and particularly relates to a phage and its use in compensating soil nitrogen fixation.

Nitrogen is one of the most important nutrients in the environment, and one of the main limiting factors for cell growth as well. About 79% of nitrogen in the air is freely available, but the vast majority of organisms need to convert nitrogen into ammonium and nitrate forms for use. It is reported that annually, the nitrogen fixation amount of legume rhizobia accounts for about 55% of the total biological nitrogen fixation amount in terrestrial ecosystems on Earth. Rhizosphere-living nitrogen-fixing bacteria can also utilize the carbon and energy sources provided by the environment to help cereal crops for nitrogen fixation. In addition, inoculation with nitrogen-fixing bacteria can alleviate the symptoms of nitrogen deficiency in wheat, enhance the salt tolerance of wheat through biological nitrogen fixation, and thus promote its growth. However, the nitrogen fixation capacity of nitrogen-fixing bacteria is limited by the cascade regulation rate between nitrogen-fixing genes. Therefore, it is necessary and urgent to invent a new soil nitrogen fixation technology which can enhance the correlation between nitrogen-fixing genes, optimize the structure of soil biological community, and break through the bottleneck of nitrogen fixation efficiency.

Recent studies have found that the bacterial nitrogen fixation process is also regulated by viruses or phages. As the most abundant organisms on Earth, phages play an important role in bacterial evolution and biogeochemical cycling. Mild phages promote horizontal gene transfer through transduction and integrate new genetic materials into the bacterial host as prophages. In marine ecosystems, viral genomes can encode auxiliary metabolic genes (AMGs) related to nitrification and denitrification, and participate in the nitrogen cycling. The participation of phages in soil nitrogen cycling affects the fixation, transformation and release of nitrogen mainly through influencing the activity and metabolism of microorganisms in soil. In addition to introducing auxiliary metabolic genes, phages can infect and kill bacteria, thus releasing organic nitrogen substances from the bacteria, which can be used by other microorganisms to participate in the nitrogen cycling process. At the same time, after phages have infected bacteria, some substrates or metabolites may be released, to promote the activities of ammonia-oxidizing bacteria and nitrobacteria in the soil, thereby promoting the nitrogen conversion process.

Phage transplantation refers to a technique that uses phages to treat bacterial infections or control bacterial populations. Soil phage transplantation is a technique that introduces phages into the soil to control or destroy specific harmful bacteria, thereby improving soil health and promoting the growth of plants. The advantages of soil phage transplantation technique are as follows: (1) this technique has high specificity, can precisely target specific microbial populations and reduce interference on non-target microorganisms; (2) this technique is environmentally friendly, because as naturally occurring viruses, phages decompose products more safely with less impact on the environment when compared with chemical pesticides or antibiotics; (3) this technique is sustainable, because phages can replicate within the target bacteria, and thus only with a lower dose of phages, a sustainable action on the target bacterial populations through carried nitrogen-fixing AMGs may be achieved.

Through relevant literature review and patent search, no publishment or acceptance of biotechnology for optimizing soil microbial nitrogen fixation system and enhancing soil nitrogen fixation efficiency by means of phage transplantation is found, and the existing method closest to the present invention is preparing microbial inoculants to promote soil nitrogen fixation, e.g., CN109970490A for preparing a nitrogen-fixing compound microbial fertilizer with high activity, improves the nitrogenase activity of root nodules and soil nitrogen fixing capacity through the synergistic effect between nitrogen-fixing bacteria in leguminous plants and purple non-sulfur photosynthetic bacteria; CN102816709A co-cultivates two different functional microorganisms in the same fermenter, so as to obtain products containing two functional bacteria and their metabolites, thus enhancing the soil nitrogen fixation; CN109824404A, CN109096008A, CN107759402A, CN106434459A and CN105925270A apply biological Azotobacter rhizobia in conjunction with waste edible fungus medium, corn cob, activated sludge and multi-source solid waste respectively for improving the soil nitrogen fixation capacity by supplementing medium trace elements necessary for crop growth and increasing the soil fertility of tailings and saline-alkali lands. The bacteria mentioned in the above patents include Bacillus, lactic acid bacterium, Saccharomyces, Acetobacter, Azotobacter, Rhizobium, etc., but the cooperative nitrogen fixation of different microbial species has not been mentioned. At the same time, there are a number of patents related to the analysis on the contribution of soil microorganisms for nitrogen fixation, e.g. CN115980306A performs isotope labelling for the nitrogen elements in soil microorganisms, measures the total nitrogen content in soil samples and the isotopically labeled nitrogen content respectively, thus obtains the total nitrogen fixation in soil after calculation; while CN108739205A achieves soil nitrogen fixation and moisture conservation by bunch planting of pea seeds in holes around the mother plants of pitaya. These methods generally consider microbial inoculants as the main methods for soil nitrogen fixation, however, the applications of phage-host synergistic nitrogen fixation pathways existing widely in soil are still seldom studied and of less concern.

Main defects of the existing technologies: The microorganisms in the existing soil microbial nitrogen fixation technologies are usually selective, and may not fully cover diversities of microorganisms in soils, resulting in limited nitrogen fixation effects. In addition, the introduction of foreign bacterial inoculants may have an impact on the original soil ecosystems, which needs to be evaluated, so as to ensure long-term ecological security, and furthermore, some microbial nitrogen fixation technologies may have the problems of unstable fertilizer efficiency or short duration, for which continuous application is required to maintain soil fertility. Even global climate change may affect the nitrogen-fixing activity and effectiveness of microorganisms. For example, changes in temperature and precipitation pattern may affect the activity and nitrogen fixation capacity of microbial inoculants.

Main reasons for the defects: There are wide varieties of microorganisms, and growth condition, nitrogen fixation capacity and interaction mechanism of each microorganism are different, and thus, in a specific environment, only some microorganisms work properly, which limits the universality and stability of nitrogen fixation effect. In addition, the interaction mechanisms of transboundary microbiomes are complex and have not been fully revealed by the current scientific research, so the potential of synergistic nitrogen fixation among microorganisms has not been fully excavated.

### SUMMARY OF THE INVENTION

Technical problem: In view of the above drawbacks in the prior art, the present invention provides a phage and its use in compensating soil nitrogen fixation.

Technical solution: Use of Klebsiella pneumoniae phage φYSZKA in compensating soil nitrogen fixation efficiency: the Klebsiella pneumoniae phage φYSZKA was collected in the China Center for Type Culture Collection (CCTCC) on Aug. 1, 2018, assigned a CCTCC NO: M 2018513, and classified as Klebsiella phage φYSZKA.

The above-mentioned soils include saline-alkali lands, pesticide-polluted soils, soybean planting soils, tea plantations or Chinese herbal medicine planting soils.

The nitrogen-fixing capacity of soils is improved by injecting Klebsiella pneumoniae phage YSZKA into the soils.

The above use also comprises the addition of biological or chemical substances that contribute to soil improvement.

The above use also comprises monitoring the change of nitrogen content of soils and adjusting the application amount of Klebsiella pneumoniae phage φYSZKA as required.

Use of Klebsiella pneumoniae phage φYSZKA in the preparation of products for compensating soil nitrogen fixation efficiency.

Working principle of the present invention: 1. Phages are one of the most abundant organisms on Earth exclusively preying on the living host bacteria; 2. The auxiliary metabolic genes (AMGs) encoded by phages can be transferred to the host bacteria through horizontal gene transfer to enhance the expression of key host function such as nitrogen fixation process; 3. After being introduced into soils, phages can infect specific nitrogen-fixing bacteria, control the number and activity of these bacteria by lytic or lysogenic pathways, and optimize the nitrogen fixation process in the soils; 4. Phages can not only reduce some excessive bacteria, but also promote the growth of other beneficial microorganisms (nitrogen-fixing bacteria, etc.) through competition and niche substitution, thereby maintaining and restoring the balance of soil microecosystem; 5. Through phage transplantation, the number and activity of nitrogen-fixing bacterial communities can be regulated, the nitrogen fixation process can be optimized, and the nitrogen fixation efficiency in soils can be improved.

Beneficial effects: 1. Improving the soil fertility. Phage transplantation can increase the content of available nitrogen in soils, promote the growth of plants, and improve the yield and quality of crops through optimizing the nitrogen fixation process; 2. Reducing the use of chemical fertilizers. Phage transplantation can improve the efficiency of nitrogen-fixing bacteria in soils, reduce the dependence on chemical nitrogen fertilizers, reduce the usage amount of chemical fertilizers in agricultural production, alleviate the pollution of water and atmosphere caused by chemical fertilizer use, and protect the ecological environment; 3. Improving the soil health. Phage transplantation promotes the balance and diversity of soil microecosystems and improves the soil health through regulating soil microbial communities. In conclusion, through combining modern biological techniques and ecological principles, the technique for compensating soil nitrogen fixation through phage transplantation provides an efficient and environmentally friendly agricultural production method, which is of great significance for the realization of sustainable agriculture.

### BRIEF DESCRIPTION OF DRAWINGS

FIG. 1 is a schematic diagram for the contribution of phage community transplantation to soil nitrogen fixation.

FIG. 2 is a TEM (transmission electron microscope) image of the phage φYSZKA;

FIG. 3 shows a one-step growth curve of the phage φYSZKA;

FIG. 4 shows the abundance of nitrogen-fixing gene nifH 45 days after microbial inoculant injection in the soil of a saline-alkali land in Nantong City, Jiangsu Province.

FIG. 5 shows the nitrogenase activity 45 days after microbial inoculant injection in the soil of a saline-alkali land in Nantong City, Jiangsu Province.

FIG. 6 shows the average concentration of organic nitrogen 120 days after microbial inoculant injection in the soil of a farmland in black soil region of Heilongjiang Province.

FIG. 7 shows the abundance of nitrogen-fixing gene nifH 50 days after microbial inoculant injection in the soil of a tea plantation in Hunan Province.

FIG. 8 shows the abundance of nitrogen-fixing gene nifK 50 days after microbial inoculant injection in the soil of a tea plantation in Hunan Province.

FIG. 9 shows the average concentration of organic nitrogen 50 days after microbial inoculant injection in the soil of a tea plantation in Hunan Province.

FIG. 10 shows the average concentration of organic nitrogen 35 days after microbial inoculant injection in a chlorobenzene pesticide-polluted soil in Anhui Province

FIG. 11 shows the relative expression level of nitrogen-fixing gene nifH 150 days after microbial inoculant injection in a Chinese herbal medicine planting soil in Longnan City, Gansu Province.

FIG. 12 shows the relative expression level of nitrogen-fixing gene nifK 150 days after microbial inoculant injection in a Chinese herbal medicine planting soil in Longnan City, Gansu Province.

FIG. 13 shows the average concentration of organic nitrogen 150 days after microbial inoculant injection in a Chinese herbal medicine planting soil in Longnan City, Gansu Province. Klebsiella pneumoniae phage φYSZKA was collected in the China Center for Type Culture Collection (CCTCC) on Aug. 1, 2018, assigned a CCTCC NO: M 2018513, and classified as Klebsiella phage φYSZKA. The address is China Center for Type Culture Collection, Wuhan University, No. 299 Bayi Road, Wuchang District, Wuhan, Hubei Province, China.

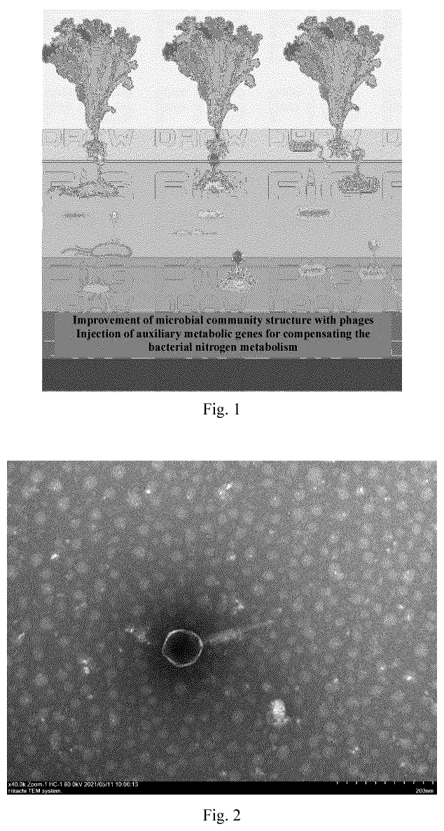

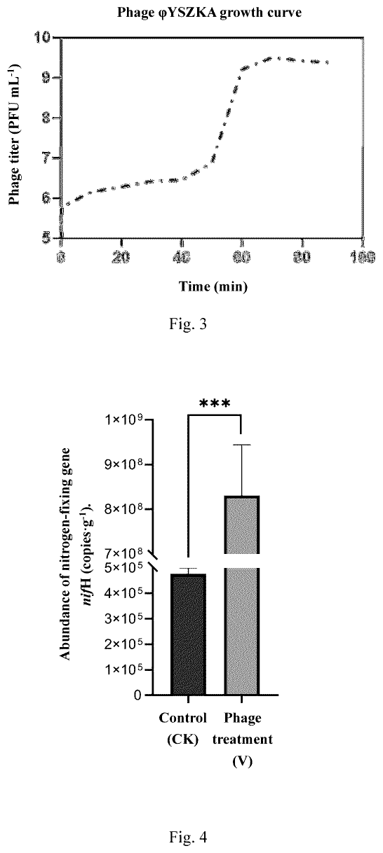

### DETAILED DESCRIPTION OF THE INVENTION

The following embodiments are not intended to limit the technical solution of the present invention in any way, and all technical solutions obtained through equivalent replacement or equivalent modification should be deemed as falling within the scope of protection of the present invention. Klebsiella pneumoniae phage φYSZKA was assigned a CCTCC NO: M 2018513 The genes nifH and nifK refer to a nitrogen-fixing functional gene carried by the phages detected in soil and a nitrogen-fixing gene carried by in-situ host bacteria, respectively.

TABLE 1
Determination of optimal multiplicity of infection (OMOI)

| Multiplicity of | | Bacterial | |
|---|---|---|---|
| infection (MOI) | Phage count | count | φYSZKA titer |
| (PFU CFU⁻¹) | (PFU mL⁻¹) | (CFU mL⁻¹) | (PFU mL⁻¹) |
| 100:1 | 1 × 10⁹ | 1 × 10⁷ | 4.1 × 10⁶ |
| 10:1 | 1 × 10⁸ | 1 × 10⁷ | 5.2 × 10⁶ |
| 1:1 | 1 × 10⁷ | 1 × 10⁷ | 1.1 × 10⁷ |
| 1:10 | 1 × 10⁶ | 1 × 10⁷ | 3.8 × 10⁷ |
| 1:100 | 1 × 10⁵ | 1 × 10⁷ | 3.6 × 10⁸ |

### Example 1

In the test potting soil, which was collected from the soil of a saline-alkali land in Nantong City, Jiangsu Province, “Rongtao No. 9” peas were planted. Basic physical and chemical properties of the soil: sand grains (sandy soil) 21.3 wt. %, soil grains 40.2 wt. %, clay grains 33.4 wt. %, pH 8.8, total nitrogen 1.6 g·kg⁻¹, water-soluble nitrogen 1.8 g·kg⁻¹, total phosphorus 1.2 g·kg⁻¹, total potassium 15.5 g·kg⁻¹, CEC 17.2 cmol·kg⁻¹.

Two groups were set for the experiment: (1) control group (CK): 3 peas were planted in each pot (with 0.5˜1 cm of soil covered on the seeds, room temperature 25±2° C.); (2) phage treatment group (V): based on the control group, 100 mL of phage suspension with a concentration of 107 PFU·mL⁻¹ was inoculated. Soil was sampled on site after the 45th day of pea growth, and the abundance of nitrogen-fixing gene nifH of the soil under treatment of the Group CK and Group V was measured: Group CK: 4.77×10⁵±2.26×10⁴ copies·g⁻¹, Group V: 8.31×10⁸±1.14×10⁸ copies·g⁻¹ (FIG. 4). Compared with the control group, the abundance of nitrogen-fixing gene nifH in soil of the saline-alkali land was increased by 3.3 orders of magnitude (p<0.05). The nitrogenase activity in the soil was measured in the treatment group and control group, which was 6.93×10⁴±6.11×10³ moles·mg⁻¹, and 3.33×10³±2.35×10² moles·mg⁻¹, respectively (FIG. 5). The results indicate that the addition of phage suspension has significant effect for enriching the microbial nitrogenase anabolism genes and promoting the soil nitrogen fixation.

### Example 2

Target soil: Farmland soil for planting “Mengdou 14” Northeast soybean in black soil region of Heilongjiang Province. Basic physical and chemical properties of the soil: pH 6.1, organic matter 28.1 g·kg⁻¹, total nitrogen 5.8 g·kg⁻¹, total phosphorus 4.1 g·kg⁻¹, rapidly available phosphorus 92.8 g·kg⁻¹, soil mechanical composition: sand grains (sandy soil) 20.8 wt. %, soil grains 45.1 wt. %, clay grains 34.1 wt. %. Organic nitrogen compounds related with soil nitrogen fixation: 834.25±27.5 mg·kg⁻¹.

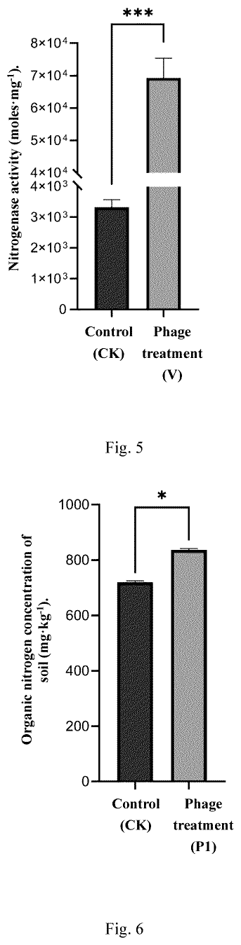

Four groups were set for the experiment: (1) control group (CK): 3 soybeans were planted in each pot (with 0.5˜1 cm of soil covered on the seeds, room temperature 20±2° C.); (2) phage φYSZKA treatment group (P1): based on the control group, 100 mL of phage φYSZKA having nitrogen-fixing function with a concentration of 10⁶ PFU·mL⁻¹ was inoculated. Soil and soybean were sampled on site after the 120th day of soybean growth, and the average concentration of organic nitrogen of the soybean soil was measured in the control group: about 720.70±2.57 mg·kg⁻¹; the average concentration of organic nitrogen of the group P1 treated with inoculated phage was increased to 836.16±5.83 mg·kg⁻¹ (FIG. 6), indicating that the phage inoculation significantly promotes the nitrogen fixation process of soil microorganisms to a certain extent, and also helps to improve the diversity and stability of soil microbial ecological functions in the black soil region.

### Example 3

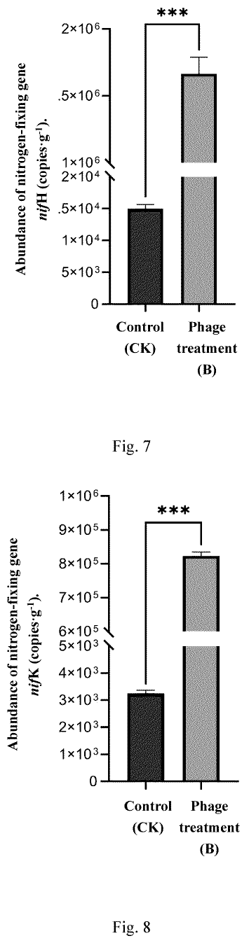

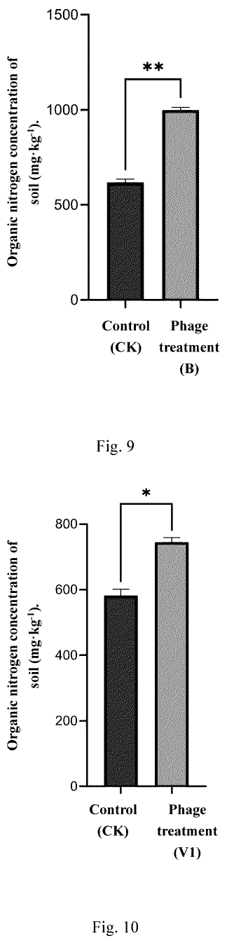

The soil for the test potted plant was collected from the soil of a tea plantation in Hunan Province. Basic physical and chemical properties of the soil: pH 4.64, organic matter 38.2 g·kg⁻¹, total nitrogen 4.1 g·kg⁻¹, total phosphorus 0.6 g·kg⁻¹, rapidly available phosphorus 35.2 g·kg⁻¹, total potassium 12.4 g·kg⁻¹, rapidly available potassium 53.2 g·kg⁻¹, soil mechanical composition: sand grains (sandy soil) 18.3 wt. %, soil grains 38.4 wt. %, clay grains 44.3 wt. %. The average concentration of nitrogen-related metabolites in zero to one meter underground soil layer was 545.2±21.3 mg·kg⁻¹. Two groups were set for the experiment: (1) control group (CK): three spinach were planted in each pot (with 0.5˜1 cm of soil covered on the seeds, room temperature 18±2° C.); (2) Phage treatment group (B): based on the control group, phage φYSZKA was applied. Soil and spinach were sampled on site after the 50th day of spinach growth, and the abundance of bacterial nitrogen-fixing gene nifH of the soil under the treatment of the Group CK and Group B was measured: 1.50×10⁴±6.61×10² copies·g⁻¹ and 1.67×10⁶±1.25×10⁵ copies·g⁻¹ (FIG. 7), and the abundance of nitrogen-fixing gene nifK of the Group CK and Group B was 3.25×10³±1.18×10² copies·g⁻¹ and 8.24×10⁵±1.11×10⁴ copies·g⁻¹ (FIG. 8), respectively. The concentration of organic nitrogen in the treatment group (Group B) (618.43±17.03 mg·kg⁻¹) with phage applied was significantly higher than that in Group CK (998.77±14.97 mg·kg⁻¹) (FIG. 9), indicating that phage transplantation can significantly regulate the effect of microbial nitrogen fixation in soil and promote plant growth.

### Example 4

Target soil: A chlorobenzene pesticide-polluted site in Anhui Province. Basic physical and chemical properties of the soil: pH 8.3, organic matter 30.3 g·kg⁻¹, total nitrogen 1.8 g·kg⁻¹, available nitrogen 28.9 mg·kg⁻¹, total phosphorus 0.2 g·kg⁻¹, available phosphorus 16.1 mg·kg⁻¹. Soil mechanical composition: sand grains (sandy soil) 9.5 wt. %, soil grains 62.8 wt. %, clay grains 27.7 wt. %. Soil nitrogen fixation related organic nitrogen compounds: 518.37±25.7 mg·kg⁻¹.

A total of four groups were set for the experiment: (1) control group (CK): three peanuts were planted in each pot (with 0.5˜1 cm of soil covered on the seeds, room temperature 20±2° C.); (2) Phage φYSZKA treatment group (V1): based on the control group, 100 mL of nitrogen-fixing functional phage φYSZKA with a concentration of 10⁶ PFU·mL⁻¹ was inoculated. Soil and peanuts were sampled on site after the 35th day of peanut growth, and the average concentration of organic nitrogen of the control group soil was measured: about 582.60±18.40 mg·kg⁻¹, and after treatment with inoculated phage V1, the average concentration of organic nitrogen was increased to 745.32±13.59 mg·kg⁻¹ (FIG. 10), indicating that the phage inoculation significantly promotes the nitrogen fixation effect of soil microorganisms, improves the soil nutrients, and enhances the diversity of soil microbial ecological functions.

### Example 5

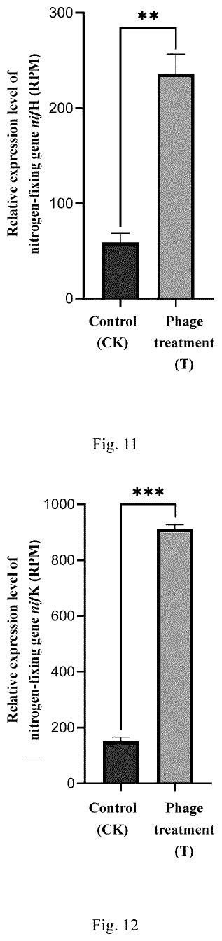

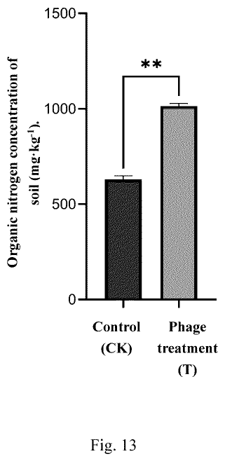

The soil for the test potted plant was collected from a Chinese herbal medicine planting soil in Longnan City, Gansu Province. Basic physical and chemical properties of the soil: pH 8.20, organic matter 32.7 g·kg⁻¹, total nitrogen 1.1 g·kg⁻¹, available nitrogen 35.2 mg·kg⁻¹, total phosphorus 0.4 g·kg⁻¹, available phosphorus 31.6 mg·kg⁻¹. Soil mechanical composition: sand grains (sandy soil) 11.2 wt. %, soil grains 65.5 wt. %, clay grains 23.3 wt. %. The average concentration of nitrogen-related metabolites in zero to one meter underground soil layer was 613.2±13.3 mg·kg⁻¹. Two groups were set for the experiment: (1) control group (CK): three tomatoes were planted in each pot (with 0.5˜1 cm of soil covered on the seeds, room temperature 18±2° C.); (2) phage treatment group (T): based on the control group, phage φYSZKA was applied. Soil and tomato were sampled on site after the 150th day of tomato growth, and the relative expression level of bacterial nitrogen-fixing gene nifH of the soil under the treatment of the Group CK and Group T was 59.00±9.75 RPM and 235.72±21.06 RPM, respectively (FIG. 11); the relative expression level of nitrogen-fixing gene nifK of the Group CK and Group T was 150.25±15.89 RPM and 910.94±15.55 RPM, respectively (FIG. 12). The concentration of organic nitrogen in the treatment group (Group T) with phage applied (1,015.08±13.90 mg·kg⁻¹) was significantly higher than that in Group CK (630.68±18.83 mg·kg⁻¹) (FIG. 13), indicating that the phage transplantation can significantly regulate the expression of host bacterial nitrogen fixing genes to promote soil nitrogen fixation, and the technology has the advantages of high broad-spectrum, low ecological risk, environmental friendliness and strong controllability, so it is a promising technology for soil nitrogen fixation and optimization.

1-5. (canceled)

6. A method for compensating soil nitrogen fixation efficiency comprising the step of adding Klebsiella pneumoniae phage φYSZKA to soil.

7. The method of claim 6, wherein the soil is for planting soybean, tea or Chinese herbal medicine.

8. The method of claim 6, wherein the nitrogen-fixing capacity of the soil is improved by injecting Klebsiella pneumoniae phage φYSZKA into the soils.

9. The method of claim 8, further comprising adding biological or chemical substances that contribute to soil improvement to the soil.

10. The method of claim 8, further comprising monitoring the change of nitrogen content in the soil and adjusting the amount of Klebsiella pneumoniae phage φYSZKA added to the soil.
